SEQUENCE LISTING (1 sequence)

SEQ ID NO: 1 - DNA, 10 bp, Artificial Sequence
  Synthetic Oligonucleotide
gtaaagtgca

(19) United States
(12) Patent Application Publication
(10) Pub. No.: US 2010/0159457 A1
(43) Pub. Date: Jun. 24, 2010

(54) SYSTEM AND METHOD FOR PRESENTING DNA BINDING SPECIFICITIES USING SPECIFICITY LANDSCAPES

(75) Inventors: Christopher Lawrence Warren, Middleton, WI (US); Aseem Z. Ansari, Madison, WI (US); Mary Szatkowski Ozers, Verona, WI (US)

Correspondence Address: Michael Best & Friedrich LLP, 100 East Wisconsin Avenue, Suite 3300, Milwaukee, WI, 53202, US

(73) Assignee: WISCONSIN ALUMNI RESEARCH FOUNDATION, Madison, WI (US)

(21) Appl. No.: 12/496,898

(22) Filed: Jul. 2, 2009

Related U.S. Application Data
(60) Provisional application No. 61/077,682, filed on Jul. 2, 2008.

(51) Int. Cl. C12Q 1/68

(52) U.S. Cl. 435/6

(57) ABSTRACT

A system and method for analyzing DNA binding specificities is provided. The potential binding motifs are compared to a plurality of DNA sequences. The DNA sequences are plotted within a specificity landscape, which provides details otherwise unavailable, relating to binding affinities and binding specificities of the motif-sequence combination.

### CROSS-REFERENCE TO RELATED APPLICATIONS

This application claims priority to provisional patent application Ser. No. 61/077,682, which was filed on Jul. 2, 2008, and is incorporated herein by reference in its entirety.

### STATEMENT REGARDING FEDERALLY SPONSORED RESEARCH OR DEVELOPMENT

This invention was made in-part with United States Government support awarded by the following agency: USDA/CSREES A073000. The United States Government may have certain rights to this application.

### FIELD OF THE INVENTION

The present invention relates to methods and systems for analyzing nucleotide sequence binding properties. In particular, the present invention relates to systems and methods for displaying DNA binding specificities.

### BACKGROUND OF THE INVENTION

Determining the sequence-recognition properties of DNA-binding proteins and small molecules has historically been a challenging endeavor, but the identification of sequence motifs has significant value. Traditionally, position-specific scoring matrices (PSSM) have been generated and manipulated for this very purpose. A PSSM can often be represented as a log-odds matrix calculated by taking the log (base 2) of the ratio of the observed to expected counts for each nucleotide in each position of the consensus motif by an algorithm like that implemented by the motif-finding program MEME. Columns and rows in the matrix correspond to the amino acids in each column and positions of the motif, respectively. A PSSM has been used to search a sequence to obtain the most probable location or locations of the motif represented by the PSSM. Additionally, PSSMs have been used to search an entire database to identify additional sequences that also have the same motif. PSSMs have struggled to be as representative as possible of the expected sites. Furthermore, the quality and quantity of information provided by a PSSM can vary for each column in the motif, which significantly affects the matches found with the sequences.

The manner in which proteins recognize specific DNA sequences is an open question of significant consequence in molecular biology. DNA recognition plays a considerable role in numerous fundamental cellular processes, including, but not limited to, DNA recombination, transcription, replication, repair, as well as the fact that DNA-binding protein defects lead to many diseases. Sifting through the rules governing protein recognition of DNA requires specific knowledge of structural details.

PSSMs, also referred to as position weight matrices (PWM), have generally been used to display nucleotide sequence specificity of DNA-binding molecules. A PSSM can be constructed once a number of DNA sequences are identified as binding to a DNA-binding molecule. Advances have been made to reduce the limitations of PSSMs for predicting and displaying DNA binding sequences. However, there remain significant limitations, such as determining how well a protein will bind a sequence predicted by PSSMs. Additionally, PSSMs assume that each position in a motif acts independently of the other positions.

Therefore, for the above reasons, it would be advantageous to use a process that more clearly represents DNA sequence motifs with interdependent positions and accurately predicts the affinity to sequences with varying levels of mismatches.

### BRIEF SUMMARY OF THE INVENTION

In at least some embodiments, the present invention relates to a method for presenting DNA binding specificities. The method includes identifying a DNA binding motif, obtaining a sample set of DNA sequences, and determining an affinity between the DNA binding motif and each DNA sequence within the sample set. The determining step is performed simultaneously for all DNA sequences. The method further includes displaying the motif-sequence binding affinity within a specificity landscape.

In at least some embodiments, the present invention relates to a system for analyzing DNA binding motifs. The system includes a micro-fabricated array for simultaneously interrogating the affinity of a DNA binding molecule with a sample set of DNA sequences, a central processing unit (CPU) for performing computer executable instructions, and a graphical user interface (GUI) for graphically displaying binding affinities. Additionally, the system includes a memory storage device for storing computer executable instructions that when executed by the CPU cause the CPU to perform a process for analyzing the array for binding affinities between a DNA binding molecule and a DNA sequence. Furthermore, the process includes: determining an affinity between the DNA binding molecule and each DNA sequence within a sample set and displaying the binding affinity within a specificity landscape.

In at least some embodiments, the present invention relates to a method for optimizing a pharmaceutical compound. The method includes the steps of identifying a pharmaceutical compound, identifying a drug target associated with the pharmaceutical compound, generating a specificity landscape for the interaction between the pharmaceutical compound and the drug target, and determining an affect of the pharmaceutical upon sequence specificity of the drug target based upon the specificity landscape. The method further includes optimizing the DNA-binding specificity of the pharmaceutical compound based upon the specificity landscape.

In at least some embodiments, the present invention relates to a method for analyzing nucleotide sequences bound by a DNA-binding molecule. The method includes the steps of identifying a DNA binding molecule, generating a set of DNA sequences, performing a cognate site identifier array for simultaneously identifying affinities between the binding molecule and each DNA sequences contained within the set; and graphically displaying the sequences in a specificity landscape based upon the number of mis-matches with the binding molecule, the position of the mismatch within the binding molecule, the particular sequence mismatch, and the binding affinities between each sequence and the binding molecule.

### BRIEF DESCRIPTION OF THE DRAWINGS

FIG. 1 is block diagram of a system for analyzing DNA binding motifs in accordance with at least one embodiment of the present invention;

FIG. 2 is a flow chart representing a method for analyzing DNA binding motifs in accordance with at least one embodiment of the present invention;

FIG. 3 is a flow chart representing a more detailed representation of various steps of the method presented in FIG. 2;

FIG. 4 is a graphical representation of a circular specificity landscape in accordance with at least one embodiment of the present invention;

FIG. 5 is a graphical representation of a linear specificity landscape based upon the same data presented in FIG. 4;

FIG. 6 is a graphical representation of a method for organizing the most likely binding motifs for a sample protein in accordance with at least one embodiment of the present invention;

FIG. 7 is a graphical representation displaying the possible motif sequences represented as a subsequence of a sample probe in accordance with at least one embodiment of the present invention;

FIG. 8 is a circular specificity landscape for the human protein p53, which binds 5′-ACATGTY-3;

FIG. 9 is a circular specificity landscape for the yeast protein msn2, which binds 5′-ARGGG-3′; and

FIG. 10 is a circular specificity landscape for the mouse protein gata4, which binds 5′-WGATAA-3′.

### DETAILED DESCRIPTION OF THE PREFERRED EMBODIMENT

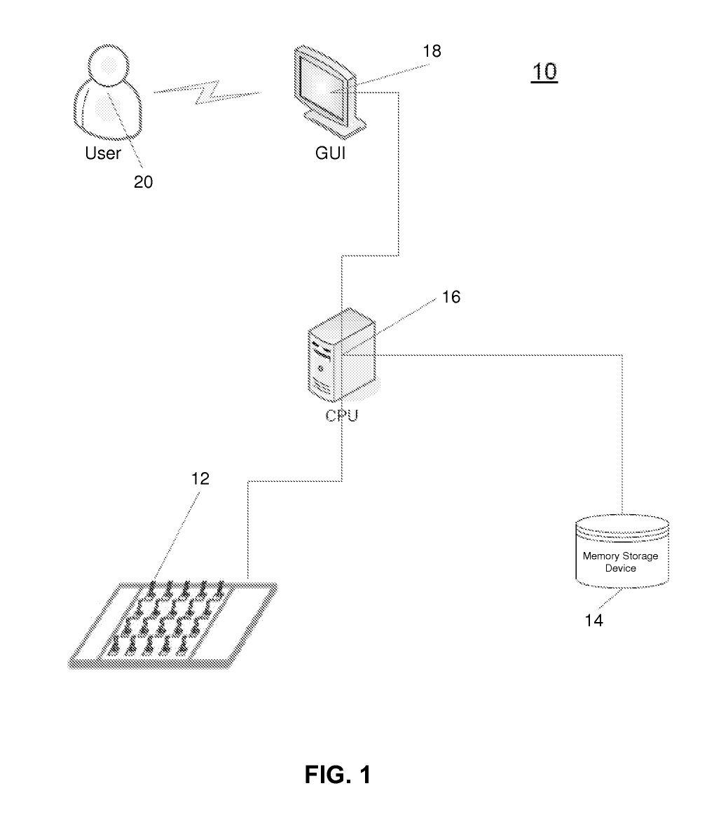

Referring to FIG. 1, a system 10 for analyzing DNA binding motifs is presented. The system 10 includes a micro-fabricated array 12, a memory storage device 14, a central processing unit (CPU) 16 and a graphical user interface (GUI) 18. The GUI enables a user 20 to view graphical representations of the analyzed DNA binding motifs. In an alternative embodiment, the CPU 16 is connected to the Internet (not shown), thereby providing a web based system for analyzing DNA binding motifs. A properly formatted data file can be dynamically created and processed through a motif-finding algorithm like MEME (http://meme.sdsc.edu/meme/intro.html), which is suitable for finding a motif within a group of sequences forming a peak, valley, or interesting position on a specificity landscape. The system 10 is configured to receive data files containing DNA sequences and associated intensities/affinities with an initial DNA binding motifs and provide the user 20 with an optimized specificity landscape.

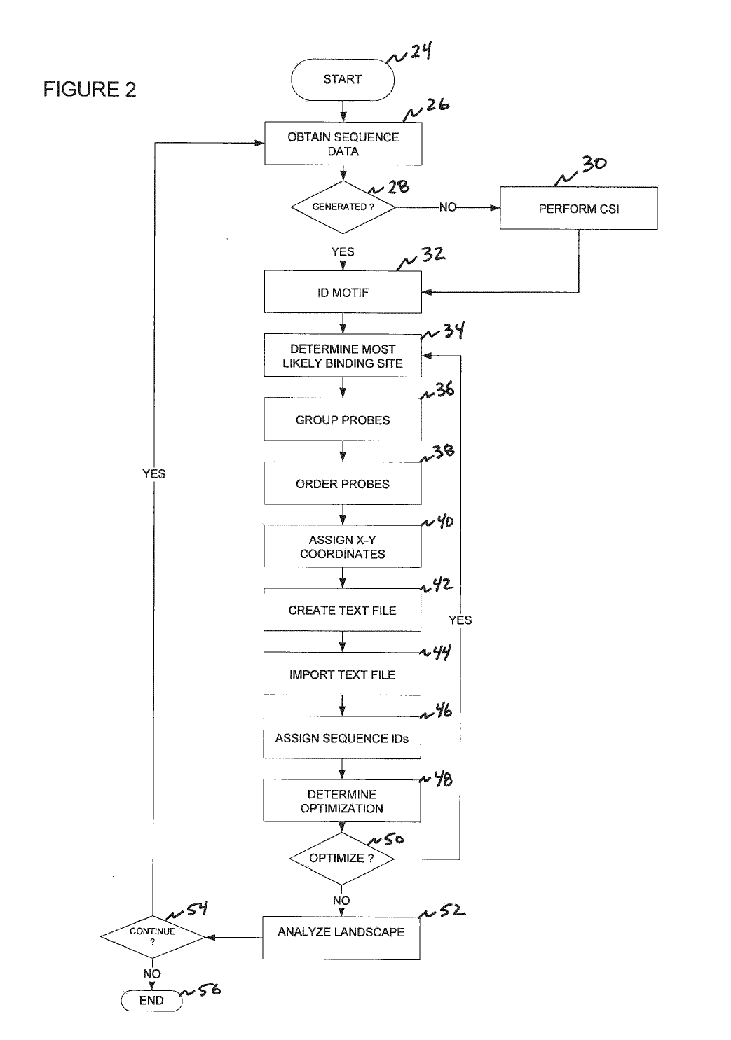

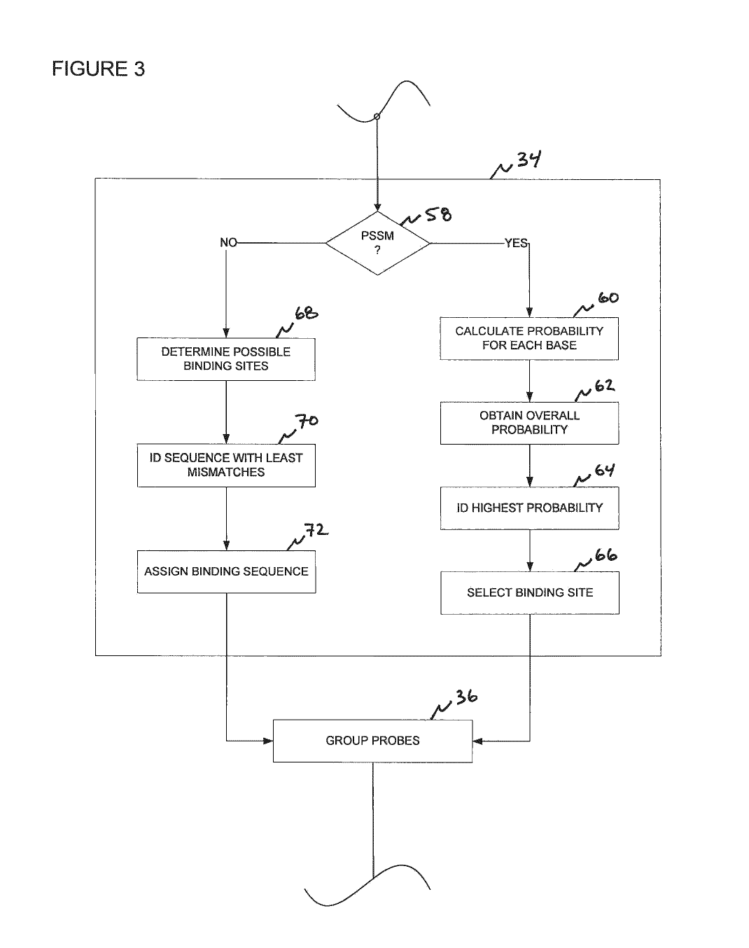

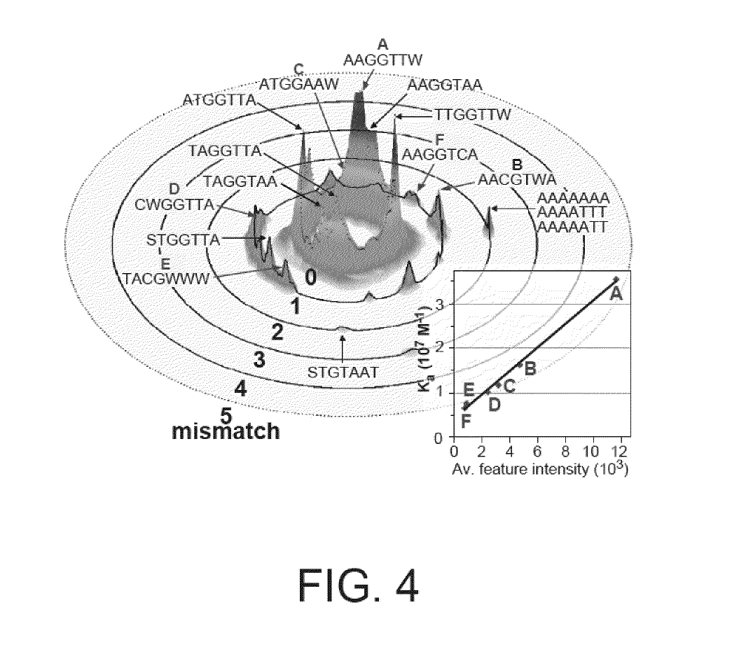

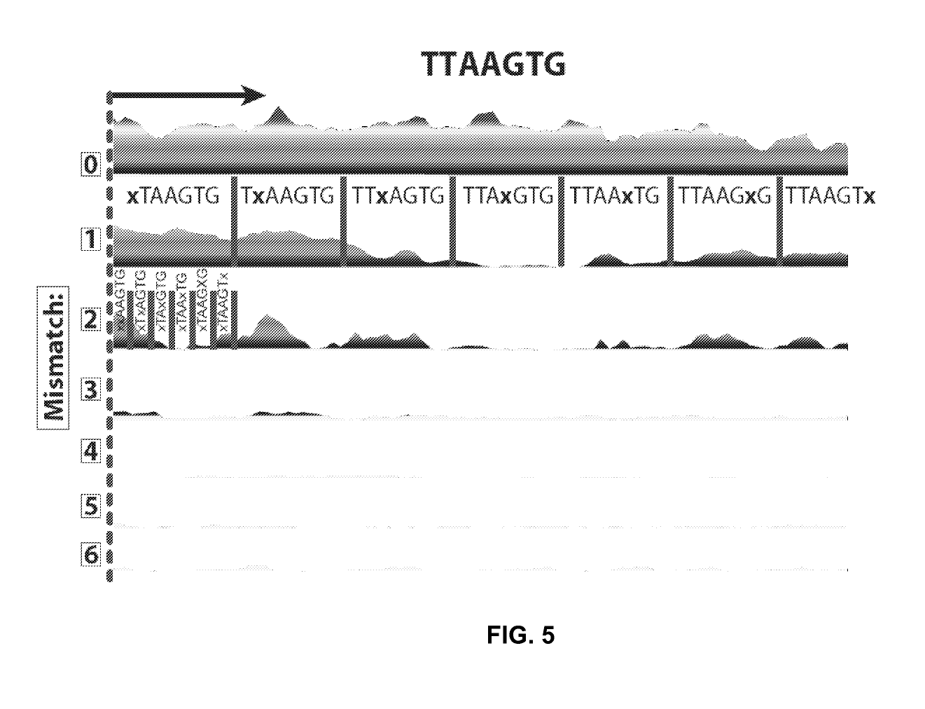

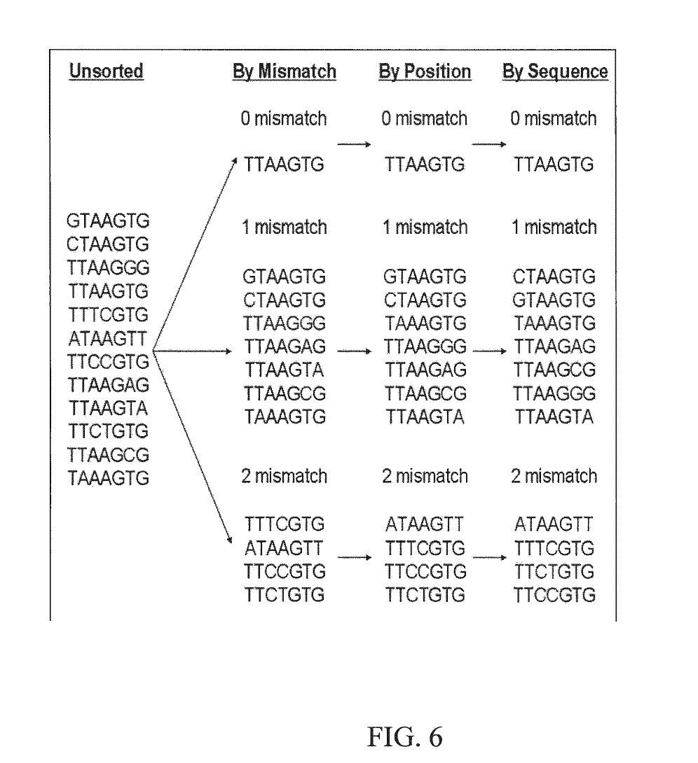

Referring to FIGS. 2-3, flow charts representing a method for presenting DNA binding specificities is presented. The system 10 is initialized at step 24. The DNA binding motif has a length between about 2 and 10 nucleotide bases. Alternatively, the molecule is greater than 10 nucleotide bases in length. DNA sequence data is obtained at step 26. Sequences that tile the entire genome of an organism or a partial desired nucleotide sequence listing to be assayed can be used as the sequence listing. If the motif-sequence affinities have not been generated at step 28, a microfabricated array is performed at step 30. In at least one embodiment of the invention a cognate site identifier (CSI) microarray is generated at step 30. Alternatively, chromatin immunoprecipitation (ChIP) microarrays and protein binding microarrays can be performed in step 30. If the motif-sequence affinity microarray has been generated then the DNA binding motif is identified at step 32, followed by determining the most likely binding site at step 34. Each of the probes is then organized by the number of mismatches, as compared to the motif, at step 36 (See FIG. 6). The specificity landscape can be visualized in a circular version (See FIG. 4) or a linear version (See FIG. 5). After the sequences are organized by the number of mismatches, the sequences are ordered within each of the mismatch groups at step 38. Each of the sequences is then assigned an X-Y coordinate at step 40.

The coordinate data is used for mapping the sequences within a specificity landscape scheme. In at least one embodiment, the coordinate data is formatted into a tab delimited text file at step 42. The text file is imported into an off-the-shelf graphical display program at step 44. A variety of software packages are available and known in the art. By example, Surfer 8 (Golden Software, Golden, Colo.) is used to graphically display the specificity landscape on a GUI. Alternatively, a graphic module (not shown) can be installed within the memory storage device 14 for seamless transition from data collection to visualization of the specificity landscape. Labels and sequence identifiers are assigned to the various peaks and valleys presented within the specificity landscape at step 46. Optimization of the specificity landscape is determined at step 48, if further optimization is requested at step 50, then step 34 is repeated. If optimization is complete, then the specificity landscape is analyzed at step 52. After analyzing the specificity landscape a decision 54 determines whether to end the process at step 56 or to repeat step 24.

Determining the most likely binding site at step 34 can be performed in a variety of methods. By example, a step 58 determines whether to use a PSSM for the motif in order to choose the most likely binding site on the probe. If a PSSM is utilized, then the probability of each DNA base is calculated for each position at step 60. The individual probabilities from step 60 are multiplied at step 62 to yield an overall probability. A subsequence is identified with the highest probability at step 64 and this sequence is selected as the binding site at step 66. If a PSSM is not utilized at step 58, then the possible binding sites are determined at step 68 (By example, see FIG. 6). The subsequence with the least mis-matches is identified as compared to a pre-determined binding motif at step 70. That subsequence is then assigned as the binding motif at step 72. The sequences are then grouped together at step 36.

In the present embodiment, a motif smaller than the lengths of the sequences is used. By example, if the nucleotide sequences are 8-mers, a 6 base pair motif can be used. This is not necessary, however; using a motif equal in length to the sequence size does not negatively impact the Specificity Landscape. A motif that is longer than the sequences used would be possible, for example in cases where the motif is matched to a similar or previously-published longer motif.

The DNA binding motif can be a single precise sequence (e.g. ACCTAG). Alternatively, the motif can be a degenerate motif, where a position in the motif is selected from more than one possible base. By example, a sequence “WCSYNV” is provided, where W=A or T, S=C or G, Y=C or T, N=any, and V=A, C, or G. Alternatively, a combination of motifs, such as “ACCTAG”, “WWGTAY”, and “GCATWC” can be represented. If a combination of motifs is used, then all motifs must be the same length. If they are not the same length, the ends of the shorter motif(s) are padded with Ns. Alternatively, the program can be re-coded to accept variable lengths in motif combinations without altering the final Specificity Landscape.

CSI arrays can display the entire sequence space for about 2-10 variable base pair positions. Additionally, CSI arrays can display the entire sequence space for more than 10 base pair positions. Data relating to the binding affinities between the binding molecule and a sample set of DNA sequences (“N-mers”) has been obtained from the CSI DNA microarrays, in which every N-mer sequence is correlated to a fluorescent intensity value. The fluorescent intensity value indicates the amount of interaction between a specific N-mer and the DNA-binding molecule of interest and is proportional to binding affinity. The affinity value is the form of an equilibrium association constant (K_{A}), or a K_{D} value may be converted to K_{A} by the relationship of K_{A}=1/K_{D}. Any biochemical/biophysical technique, other than DNA microarrays, can be used to obtain binding affinities, but these affinities must be related to a specific DNA sequence to be appropriate input for the specificity landscape.

A partial DNA sequence list is acceptable for input into a specificity landscape. However, progressively fewer sequences lead to progressively rougher, and often less reliable, specificity landscapes. By example, all possible 8-mer DNA permutations of the 4 DNA nucleotides ACGT are 4⁸ which equal 65,536 different DNA sequences. A specificity landscape can be generated using 2000 of the 65536 possible 8-mer DNA sequences, but this will lead to more disjointed landscapes and less information. Furthermore, if this partial sequence list is biased towards certain sequences, the specificity landscape will also be biased towards these sequences. Specificity landscapes can be generated with as few as two DNA sequences, but the analytical value provided by a specificity landscape is not significant unless the sample set of sequences is significant. Alternatively, sequences that tile the entire genome of an organism can be analyzed, which can include greater than 100,000 sequences.

The freely available internet-based program MEME (http://meme.sdsc.edu/meme/intro.html), or an alternative program, can be used to generate a motif from the highest intensity (affinity) sequences within a sample set. However, any motif can be used, and in certain cases a randomly generated motif may be utilized when no binding motif is predicted from MEME or an equivalent program. Ideally, motifs having between 5 and 15 base pairs are utilized, but motifs of 3 by or less can be used to produce specificity landscapes, the resulting Landscape will be less informative because biological motifs average 5-10 by in length and a motif of 3 by or less has a greater chance of being random. A specificity landscape can be generated for greater than 15 base pair motifs.

Referring to FIG. 4, a circular specificity landscape example is provided. For the circular version, all DNA probes that have a binding site exactly matching a motif are placed on a ring with a radius of 1. These probes are evenly distributed throughout the ring based on the ordering of nucleotide sequences. This is repeated for all DNA probes containing a binding site with one mismatch to a motif, but placed on a circle with a radius of 2. This is done for each mismatch group, where the radius of the circle is one greater than the number of mismatches to the most similar motif. Redundant sequences are removed from the circular landscapes in order to reduce the density of points in the outer rings of the landscape. By example, the second mismatch ring of a 5 base pair consensus sequence, the data can be sorted by mismatch positions 1-2, followed by 1-3, 1-4, etc. Redundant sequence mismatch 2-1 is removed, as it was already provided. Each specificity landscape provides significant details relating to DNA sequences recognized by DNA binding molecules. In particular, the specificity landscape provides the relative affinity of a particular binding molecule to every DNA sequence that is simultaneously assayed.

The specificity landscape of FIG. 4 is based upon data obtained from a CSI array, in which B-form DNA conformers were displayed. This particular approach is an example of the data input to the specificity landscape algorithm. In particular, the present approach provides a comprehensive and unbiased understanding of the sequence-specificity of DNA binding molecules. Structural DNA variants are all examined to explore the importance of DNA structure on cognate site recognition by these ligands (data not shown). This particular example examines sequences and structural preferences of a small molecule polyamide (ImImPy*Py-γ-PyPyPyPy-β-Dp) designed to recognize the sequence 5′-W-W-G-G-W-W-W-3′ (W=A or T). The polyamide shows a decreasing preference for increasingly unusual DNA conformers. This particular polyamide prefers double-stranded DNA. The more distorted, or non-duplex, the DNA becomes the less affinity the polyamide has for that particular DNA sequence. Based upon previous knowledge of this polyamide, the specificity landscape unexpectedly uncovered unique insight into the polyamide relating to sequence and structural specificity of the polyamide-DNA interactions. In particular, for high affinity duplex sequences, the corresponding unusual DNA conformers (non-B-form conformers) still exhibit appreciable binding relative to the duplex. The relative affinity of a particular sequence is represented by the height of the peak. The landscape provides a valuable tool to researchers looking for both the consensus sequences as well as those with the highest affinity.

Each sequence on the array is given a z-score which indicates the probability of a sequence being preferentially bound by the polyamide. As such, each sequence on the array has an intensity denoting the affinity of the polyamide to the sequence. The z-score is calculated using Equation Set 1. The sequences in the highest z-score bins, or with the highest intensities, fit the 5′-W-W-G-G-W-W-W-3′

Z=|intensity−average|/standard deviation Equation Set 1:

(W=A or T) motif, for which the polyamide was specifically designed. However, there are differences in binding with variations of the consensus motif. By example, both AAGGTTW and TAGGTAA are represented in the first ring, yet have significant differences with their peaks. The former binding motif has a much greater and more significant peak (See A). Because all the peaks within the innermost ring (Ring 1) are not the same height, embodiments of the present invention demonstrate that the polyamide does not bind all the permutations of the consensus motif (WWGGWWW) with equal affinity. The CSI landscape indicates the importance of each position in the consensus motif. For this polyamide, the most flexible positions are 1 and 3 since the highest peaks in the one mismatch ring correspond to 5′-SWGGWWW-3′ (S=C or G) and, unexpectedly, 5′-WWNGWWW-3′ (G>>C>W>0). This important sequence binding detail can not be seen in Logos or in a corresponding PSSM, which are limited by the lack of intensity for each permutation of the consensus motif and inability to indicate interdependencies between positions within the motif. By accounting for the interdependencies between positions within a motif, embodiments of the present invention provide an advantage to analyzing DNA binding molecule affinities, and provides details for recognition properties of the DNA ligands.

With respect to the present polyamide, the CSI landscape provides significant and valuable information for analysis of DNA binding molecules. In particular, the CSI specificity landscape provides details relating to polyamide-DNA interactions. By example, the tested polyamide tolerates mutations at positions 1 and 3, while it prefers a T at position 5, and shows appreciable binding to non-B-form DNA bearing high affinity sequences. The current embodiment is not limited to analysis of synthetic DNA ligands, but can probe the sequence preferences of the DNA-binding proteome for any organism. By example, a sample of different organisms and proteins can be displayed using specificity landscapes, including Human (p53 and c-abl), Mouse (nkx-2.5 and gata4), Drosophilia (dfd and abdB), and Yeast (msn2). In addition, the present embodiment can be used to develop new sequence-specific DNA ligands or to evaluate how small changes to current ligands affect their specificity and affinity to DNA. This translates directly to facilitating the creation of synthetic molecules that target and regulate gene networks with high precision.

Specificity landscapes provide an alternative method of presenting DNA binding data. Each landscape displays the relative affinity of a particular binding molecule for every DNA sequence assayed simultaneously. A specificity landscape displays DNA sequences plotted on a series of concentric rings where each DNA sequence represented on the center ring perfectly matches the binding motif while those on the second ring have one mismatch, the third ring is made of all sequences with two mismatches, etc. The relative affinity of a particular sequence is represented by the height of the peak, the greater relative affinity the higher the peak.

Referring to FIG. 5, a linear specificity landscape is provided that correlates to the circular specificity landscape provided in FIG. 4. Each level of sequence mismatch is presented in a separate panel. Sequences matching the consensus sequence are in the first panel, followed by single mismatches, two mismatches, three mismatches, four mismatches, and five mismatches. For this particular linear landscape, the binding motif is 5′-TTAAGTG-3′. In order to reduce the density of outer ring points within a circular landscape, redundant sequences are removed. Redundancy displayed in linear landscapes can be removed or maintained. Preferably, the redundant points are maintained, thereby providing a pattern for easier orientation between linear panels and analysis by a user. For the linear landscapes, each mismatch group is given its own panel. The probes in that group are distributed evenly across the panel based on the ordering scheme employed.

Referring to FIG. 6, a graphical example of step 36 (See FIG. 2) is depicted. The most likely binding sites derived from a sample input file for Protein X is shown. The consensus sequence, “TTAAGTG”, is compared with the DNA binding molecules of equal length. The binding site list is sorted first by number of mismatches, then by position of the mismatch(es), and finally by sequence (A, then T, then C, then G). The ordering of the DNA sequences can affect the visibility of peaks and valleys displayed in the specificity landscape. Regardless of which method is used to determine the most likely binding site on the probe, the probes are sorted by the number of mismatches of the binding site as compared to the motif (or most similar motif if there are multiple motifs). This determines on which ring (circular version of the specificity landscape) or panel (linear version) the probe will be placed. In an alternative embodiment (not shown), the sequence ordering can be altered. By example, the sequences can be ordered first by the position of the mismatch, then by the number of mismatches, followed by the sequence mismatch. Altered ordering can be dynamically altered by the system 10 based upon a user's preference and analysis methodology.

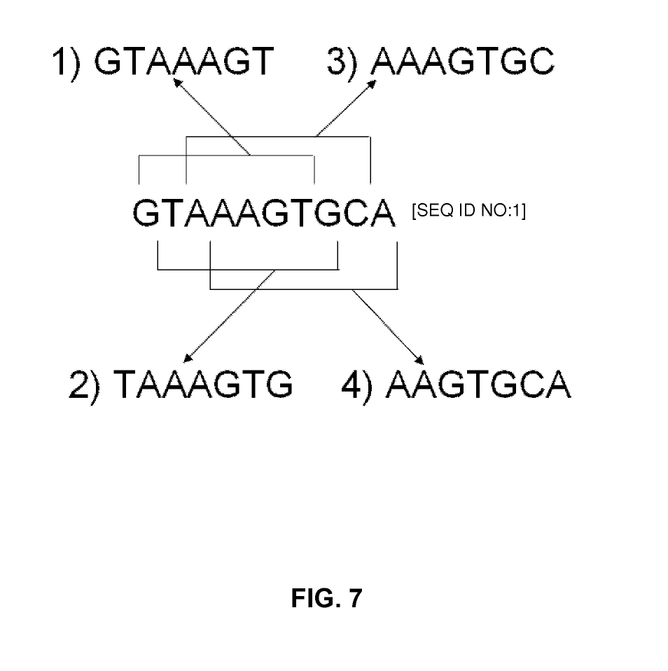

Referring to FIG. 7, a graphical example of step 38 (See FIG. 3) is depicted. The motif is 7 base pairs and the DNA probes are 10-mers (10 variable positions). There are a total of 4 overlapping possible binding sites in this variable region. The most likely binding site is the subsequence with the fewest number of mismatches as compared to the pre-determined motif. By example, a predetermined motif can be MEME derived.

A tab delimited text file is created when using separate graphical display software. Import the file into Surfer 8 landscaping display program (Golden Software, Golden Colo.). This particular program allows for smoothing the peaks and providing color variations in the landscape peaks. Alternately, a graphing module can be added to system 10. An exemplary graphing module includes use of MATLAB (MATLAB 7, The Mathworks Inc., Natick, Mass.) for parsing data and displaying a specificity landscape. Smoothing, as opposed to plotting the raw data, has the advantages that peaks are more easily discerned and noise (e.g. variation from identical binding sites on different probes) is reduced. Many different smoothing algorithms exist, by example the ‘minimum curvature’ algorithm can be utilized to avoid over-smoothing. This smoothing algorithm is dynamically included within the computer executable files of the memory storage device 14.

Text labels can be automatically provided for interesting and/or significant peaks or valleys. A variety of methods can be employed for identifying peaks and valleys. One exemplary methodology identifies the average intensity or affinity of a specific ring as well as the associated standard deviation. Any sequence within the ring having an affinity/intensity value above or below the average can be labeled automatically. Alternatively, sequences that differ by a predetermined value can be highlighted for analysis. Additionally, edges of labeled peaks where the value rises above (or below for valleys) the standard deviation are labeled and archived.

A motif(s) can be optimized followed by re-running a specificity landscape. Based on the number of peaks that occur outside the central motif-matching ring or valleys that occur within the central ring, a new and more accurate motif(s) can be determined. This can be performed manually by examining the specificity landscape with sequence labels attached and subjectively deciding whether there are too many high-intensity peaks in an outer ring or too many valleys in the central ring. However, this optimization step can be automated and incorporated into a computer executable. First the smoothing algorithm is coded into the script. The sequences that represent any regions significantly lower than the average height in the central ring are then aligned and removed from the motif(s). Sequences from regions in the outer rings that are at least 75% (or any user defined percentage) of the average central ring height are aligned and included in the motif(s). This can be done iteratively until a solution is achieved or a user defined number of iterations are reached. In fact, a variation of the specificity landscape in which just an optimized motif(s) is returned to the user without any landscape image could be developed as an improved version of MEME or any other motif discovery software.

The cognate site identifier array is a high through put approach for providing a comprehensive profile of the binding properties of DNA-binding molecules. CSI arrays display every permutation of a duplex DNA sequence on a microfabricated array. The CSI is a standard type array where each square (microarray feature) is assigned a specific nucleotide sequence, which includes a linker and a palindromic sequence with a 3-5 nucleotide turn in the middle. This palindromic sequence forms a double-stranded DNA region comprised of, for example, 8 base pairs that represents a specific permutation of an 8mer sequence. The central region of the sequence is buried, by example all or a subset of, 8 base pairs for a given 8-mer sequence. Approximately one million of the same sequences are provided for each square, each being one of the possible 8-mer sequences. A fluorescently labeled compound or antibody is included. An intensity for each feature is obtained and an affinity value is obtained for each sequence for the particular 8-mer. Each sequence is assigned an X-Y coordinate, the Z coordinate is the intensity value.

CSI Example 1: Nkx-2.5 (Nk2 transcription factor related, locus 5)

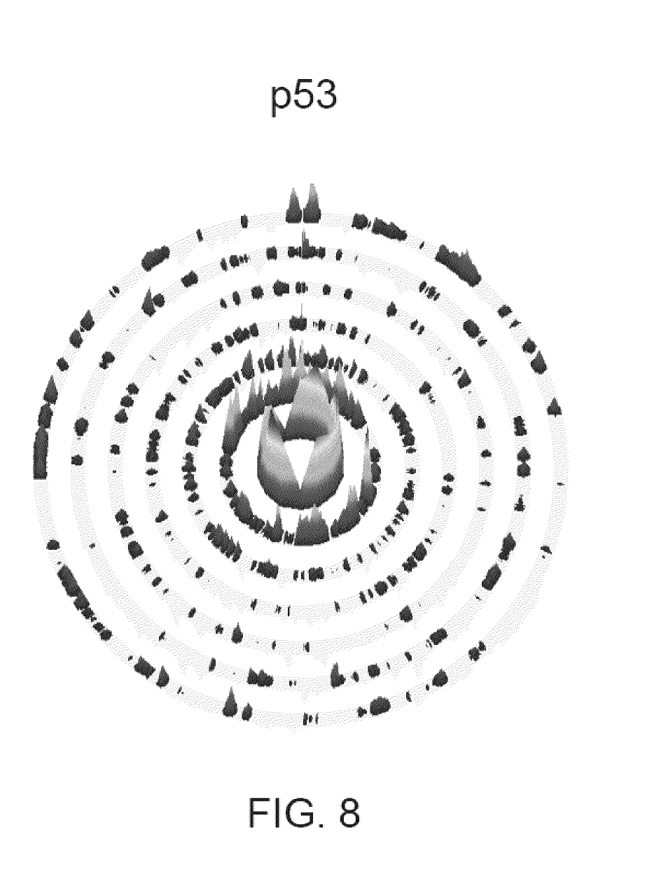

The Nkx-2.5 is the earliest heart lineage marker expressed in precardiac cells during mammalian development and has been linked to familial congenital heart disease. In order to accurately predict the DNA binding specificity of Nkx-2.5 the relative affinity for every possible 9-mer DNA sequence was assayed by CSI. When a specificity landscape of the sequence predicted by the Logo “TTAAGTG” is prepared using the probabilities from a position weight matrix (PWM) instead of CSI intensities it illustrates one of the inherent limitations of PWMS, which is compression of data and loss of potentially important subtleties. A CSI specificity landscape for motif “TTAAGTG” is prepared displaying the relative intensities of Nkx-2.5 for all possible 9-mers, which indicates that all sequences identical to this motif bind well (See FIG. 8A). Based upon the high intensity binding in the second ring, it is clear that the motif is too restrictive (See FIG. 8B). By example, when a specificity landscape is prepared for the “AAGTG” motif (not shown), low intensity sequences are found in the center ring. By displaying specificity landscapes with high center ring intensities and low relative intensities in the outer rings, greater detail regarding the motif-sequence binding can be ascertained. By example, based upon the specificity landscape, the DNA binding motif of the present Nkx-2.5 sequence is “TNAAGTG and NTAAGTG”. Therefore, the specificity landscape is advantageous over the PWM because it clearly represents the motifs with interdependent positions, displays sufficient sequence space to confidently yield a motif, accurately predicts affinity to sequences with multiple mismatches and clearly represents the relative affinity for a binding molecule to every DNA sequence tested.

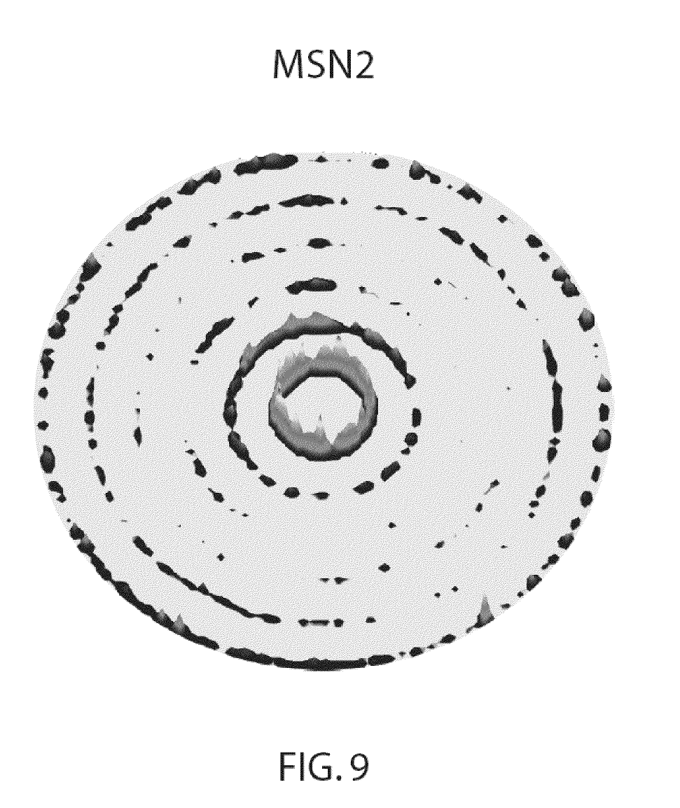

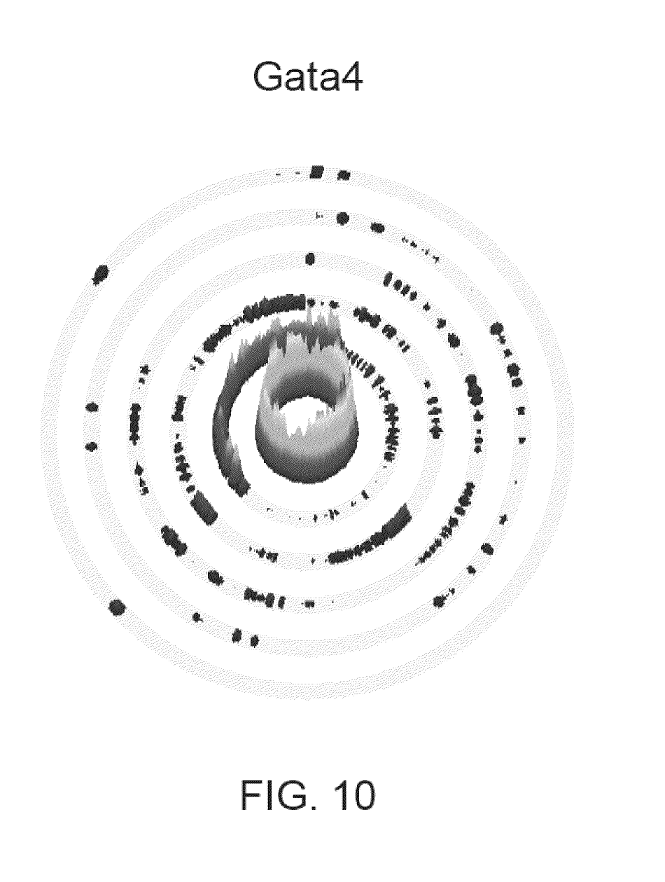

Referring to FIGS. 8-10, alternative examples of specificity landscapes are provided. The specificity landscape for human protein p53, which binds 5′-ACATGTY-3 is provided in FIG. 8. The specificity landscape for yeast protein msn2, which binds 5′-ARGGG-3′ is provided in FIG. 9. The specificity landscape for mouse protein gata4, which binds 5′-WGATAA-3′, is provided in FIG. 10.

Utilization of the CSI array and specificity landscape provides a comprehensive and unbiased understanding of the sequence-specificity for developing DNA binding molecules. Specifically, the creation and ability to program key transcriptional regulators with great precision, including their DNA recognition properties can be obtained by various embodiments of the present invention. These processes can be used to design synthetic molecules that target and regulate the expression of desired genes. In addition to CSI array data, specificity landscapes can be generated from chromatin immunoprecipitation (ChIP) microarrays and protein binding microarrays.

In at least one embodiment of the present invention, the specificity and affinity of DNA ligands are queried by using a duplex DNA microarray that displays the entire sequence space (See FIG. 4). The DNA probes are composed of 15 base pair duplex hairpins and each spatially resolved feature on the array bears a unique sequence permutation. Incubating a labeled DNA-binding molecule with all the probes on the CSI array provides the complete sequence recognition profile for the particular DNA ligand. The structural variants of the DNA binding molecules explore the importance of DNA binding molecules. This comprehensive survey of structure and sequence space, which is performed by various embodiments of the present invention, are not performed by presently available biochemical methodologies. Furthermore, the CSI approach utilizing unimolecular probe design permits repeated usage of the array with out loss of information. This, in part, is an advance to CSI micro-fabricated arrays, which allow for a rapid, cost effective platform with which to comprehensively test the structural as well as sequence recognition properties of DNA binding molecules.

In an alternative embodiment, the present invention includes a method for optimizing a pharmaceutical compound. A pharmaceutical compound is identified for detailed analysis along with the drug target associated with the pharmaceutical compound. A specificity landscape is generated that provides details of the interaction between the pharmaceutical compound and the drug target. Based upon the specificity landscape generated for the pharmaceutical interaction, an affect of the pharmaceutical upon sequence specificity of the drug target is generated. Based upon the specificity landscape, the DNA-binding specificity of the pharmaceutical compound is optimized.

Therapeutics targeting specific protein-DNA interactions in disease is an under-studied area of drug design. Specificity landscapes within the present embodiment can determine how drug interactions affect the sequence specificity of the drug bound to DNA. By example, the p53 transcription factor and the estrogen receptor are drug targets that can be analyzed by specificity landscapes for optimizing pharmaceuticals designed to interact with these particular transcription factors. Lead compounds can be altered to optimize DNA-binding specificity, and the specificity landscape is a diagnostic tool used within this process.

In an alternative embodiment, specificity landscapes are used to predict how a therapeutic, such as a drug or alternative chemical, affects DNA binding specificity of a drug target (protein, aptamer, etc) in individual patients bearing single nucleotide polymorphisms (SNPs). An individual or a sub-set of the population may be presented with a specific SNP. Identifying the SNP and performing a CSI specificity landscape provides data as to the binding affinities between the sequence presented with the SNP and the normal sequence. Comparing the interactions between the normal sequence and the SNP sequence can lead to designing synthetic drugs designed specifically for the sequence presented with a SNP.

In an alternative embodiment, specificity landscapes are used to determine how a therapeutic can bind to unexpected DNA sites and trigger aberrant gene regulation, which can be predictive of potential drug side effects.

In yet another alternative embodiment, approximately 6% of the human genome encodes DNA binding transcription factors but the comprehensive binding profiles of these factors are only known for a small subset. Specificity landscapes strengthen the data for transcription factor databases such as TRANSFAC. One additional application determines how a naturally occurring small molecule (such as cAMP) or a potential drug that interacts with a transcription factor affects its sequence specificity. This would be achieved by comparing a specificity landscape of the transcription factor alone to that of the transcription factor with the compound.

In an alternative embodiment, specificity landscapes are used to uncover neighboring effects of a binding motif as the sequence around the binding motif can often affect affinity even though the protein makes no direct contact with those base pairs. Specificity landscapes can also reveal biologically-relevant and lower affinity binding motifs that are often obscured by the primary binding motif(s).

In an alternative embodiment, specificity landscapes can improve standard motif finding algorithms, such as MEME or MDScan for high-throughput data analysis. Specificity landscapes could be applied to in vitro experiments such as CSI, SELEX, and fluorescence anistropy (in a mid to high-throughput microwell format). However, for more complicated in vivo experiments like chromatin immunoprecipitation with microarray analysis (ChIP-chip), it will require careful consideration of all possible scenarios including hugely disparate sized probe sequences (e.g. sequences representing ChIP-chip peaks). To avoid false positives and negatives in ChIP-chip, careful optimization of specificity landscapes will need to be done to allow for cases where some high affinity probes have no binding site or multiple binding sites and where low affinity probes have a binding site that normally yields high affinities. Despite these considerations, specificity landscapes are applicable for ChIP-chip data.

The following documents are hereby incorporated by reference in their entirety, herein. Additionally, all the documents directly cited within the documents cited below, are hereby incorporated by reference in their entirety herein.
1) C. L. Warren et al., Defining the sequence-recognition profile of DNA-binding molecules, Proceedings of the National Academy of Sciences 103, 867 (2006).
2) S. J. Maerkl, S. R. Quake, A systems approach to measuring the binding energy landscapes of transcriptional factors, Science 315, 233 (Jan. 12, 2007).

It is specifically intended that the present invention not be limited to the embodiments and illustrations contained herein, but include modified forms of those embodiments including portions of the embodiments and combinations of elements of different embodiments as come within the scope of the following claims.

1.-16. (canceled)

17. A method for optimizing a pharmaceutical compound, comprising the following steps:
identifying a pharmaceutical compound;
identifying a drug target associated with the pharmaceutical compound;
generating a specificity landscape for the interaction between the pharmaceutical compound and the drug target;
determining an affect of the pharmaceutical upon sequence specificity of the drug target based at least in part upon the specificity landscape; and
optimizing the DNA-binding specificity of the pharmaceutical compound based at least in part upon the specificity landscape.

18. The method according to claim 17, wherein the optimizing includes altering the chemical structure of the pharmaceutical compound to improve its specificity.

19. A method for identifying a pharmaceutical side affect in a human, comprising the steps of:
identifying a pharmaceutical compound;
obtaining a human genome comprising a sample set of DNA sequences;
determining an affinity between the compound and each DNA sequence within the sample set;
generating a specificity landscape based at least in part upon the determined affinity;
identifying compound-DNA binding based at least in part upon the specificity landscape; and
identifying pharmaceutical side-effects caused at least in part by aberrant gene regulation based at least in part upon compound-DNA binding.

20. A method for analyzing nucleotide sequences bound by a DNA-binding molecule, comprising the following steps:
identifying a DNA binding molecule;
generating a set of DNA sequences;
performing a cognate site identifier array for simultaneously identifying affinities between the binding molecule and each DNA sequences contained within the set; and
graphically displaying the sequences in a specificity landscape based at least in part upon the number of mis-matches with the binding molecule, the position of the mismatch within the binding molecule, the particular sequence mismatch, and the binding affinities between each sequence and the binding molecule.
